(19) United States
(12) Patent Application Publication
(10) Pub. No.: US 2026/0358325 A1
(43) Pub. Date: Nov. 12, 2026

(54) TOILET ROLL

(71) Applicant: Nippon Paper Crecia Co., Ltd., Chiyoda-ku, Tokyo (JP)

(72) Inventors: Yasunobu OOKA, Chiyoda-ku, Tokyo (JP); Hikaru SATO, Chiyoda-ku, Tokyo (JP); Yuki FUJITA, Chiyoda-ku, Tokyo (JP); Misa SHIMIZU, Chiyoda-ku, Tokyo (JP); Riho IINO, Chiyoda-ku, Tokyo (JP)

(73) Assignee: Nippon Paper Crecia Co., Ltd., Chiyoda-ku, Tokyo (JP)

(21) Appl. No.: 19/470,193

(22) PCT Filed: Mar. 21, 2024
(86) PCT No.: PCT/JP2024/011066

§ 371 Date: Nov. 26, 2025

(30) Foreign Application Priority Data
Mar. 31, 2023 (JP) 2023-058537
Aug. 23, 2023 (JP) 2023-135874

(51) Int. Cl. G01N 33/34; B32B 3/30; B32B 29/00; D21H 27/30; G01N 3/40

(52) U.S. Cl. CPC G01N 33/34; B32B 3/30; B32B 29/005; D21H 27/30; G01N 3/405; B32B 2250/02; B32B 2250/03; B32B 2250/26; B32B 2307/718; B32B 2307/72; B32B 2307/7376

(57) ABSTRACT

A toilet roll includes two-ply toilet paper wound into a roll shape, wherein the roll has a softness of wound roll of not less than 0.5 mm and not more than 4.8 mm, a wound diameter of not less than 120 mm and not more than 150 mm, the toilet paper contains a temporary wet strength agent, the basis weight per ply is not less than 16 g/m² and not more than 26 g/m², the ratio of TS750 of the ply on the roll outer surface side to TS750 of the ply on the roll inner surface side is not less than 0.8 and not more than 1.7, the embossing is provided on the ply on the roll outer surface side and includes a linear first emboss, and the area ratio of the first emboss is not less than 35% and not more than 93%.

### TECHNICAL FIELD

The present disclosure relates to a toilet roll in which a two-ply toilet paper, to which embossing has been applied, is wound.

### BACKGROUND

Commercially available toilet rolls have been increasing in wound length, and in addition, high-end products are also required to have high quality.

In particular, high-end products are required to have a favorable tactile sensation and aesthetic appearance, as well as resistance to tearing when used with warm-water washing toilet seats. Furthermore, by increasing the wound diameter while maintaining the softness of the roll, a toilet roll with a more luxurious feel can be achieved.

In order to provide toilet paper that is resistant to tearing when used with warm-water washing toilet seats, it is possible to add a temporary wet strength agent in the papermaking process, thereby making the toilet paper (sheet) become prone to tearing even when used with warm-water washing toilet seats.

As prior art documents for toilet paper suitable for use with warm-water washing toilet seats, for example, PTL 1 discloses a two-ply toilet paper having a basis weight per ply of not less than 18 g/m² and not more than 23 g/m², a specific volume of not less than 5.0 and not more than 6.5, and a machine direction wet tensile strength in four plies of not less than 90 gf/25 mm and not more than 130 gf/25 mm, with the wound length per roll of not less than 30 m, and when the machine direction dry tensile strength as defined in JIS P 8113 is expressed as DMDT and the lateral dry tensile strength as DCDT, the ratio DMDT/DCDT in two plies is not less than 2.0 and not more than 3.5.

Further, PTL 2 discloses a toilet roll in which a one-ply toilet paper having a plurality of irregularities, with one side being a convex portion and the corresponding opposite side being a concave portion, is wound into a roll, the wound length is 105 to 210 m, the wound diameter is 100 to 140 mm, the roll weight excluding the core per 114 mm roll width is 225 to 485 g, the core per 114 mm roll width is 3.2 to 6.0 g, the outer diameter of the core is 25 to 48 mm, and the winding density of the roll is 0.17 to 0.35 g/cm³. It is also described that the irregularities are embosses.

### CITATION LIST

### Patent Literature

PTL 1: JP 2015-126771 A
PTL 2: JP 2018-000728 A

### SUMMARY

### Technical Problem

Meanwhile, in toilet paper, in order to enhance the aesthetic appearance of embosses, it is necessary to firmly provide embosses (increase the emboss depth), but if the emboss is made deeper, in the case of single embossing, the difference in tactile sensation between the outer surface side and the inner surface side is increased.

On the other hand, double embossing tends to increase bulk, but in patterns such as FIG. 2 of PTL 2, the bulk may become excessive. In addition, since the emboss is dot-shaped, the corners around the emboss portions tend to become sharp, and even with double embossing, the difference in tactile sensation between the outer surface side and the inner surface side may become large.

Of course, if the emboss is made shallower, the difference in tactile sensation between the outer surface side and the inner surface side becomes smaller, but in exchange, the aesthetic appearance of the emboss deteriorates. If, in that state, the aesthetic appearance of the emboss is enhanced by bonding the plies with glue, the paper becomes stiff due to the influence of the glue. Furthermore, a sheet containing a temporary wet strength agent feels stiff, and the tactile sensation is even worse than that of ordinary toilet paper.

For these reasons, in toilet paper and toilet rolls suitable for use with warm-water washing toilet seats, it has been difficult to satisfy all of resistance to tearing, smoothness, aesthetic appearance and sense of volume of the emboss, as well as appropriate wound length and wound diameter of the roll, and softness of the roll.

The present disclosure has been made in view of such circumstances, and an object thereof is to provide a toilet roll in which the toilet paper contains a temporary wet strength agent and is therefore suitable for use with warm-water washing toilet seats, is resistant to tearing, excels in smoothness, aesthetic appearance and sense of volume of the emboss, and further, the roll has an appropriate wound length and wound diameter, and also exhibits favorable softness.

### Solution to Problem

The inventors have conducted intensive studies and, in a toilet roll in which a toilet paper, formed by laminating two-ply or three-ply sheets to which embossing has been applied and which have been bonded with water and/or glue, is wound into a roll, by setting the softness of wound roll and wound length of the roll each within a predetermined numerical range, causing the toilet paper to contain a temporary wet strength agent, and further, by setting the TS750 of the roll outer surface side and roll inner surface side of the toilet paper and their ratio, the basis weight of the toilet paper, and the area ratio of the first emboss included in the embossing in the toilet paper each within a predetermined numerical range, it is possible to provide a toilet roll suitable for use with warm-water washing toilet seats, resistant to tearing, excelling in smoothness, aesthetic appearance and sense of volume of the emboss, and further, the roll has an appropriate wound length and wound diameter, and also exhibits favorable softness, thereby completing the present disclosure. That is, the present disclosure provides the following.

(1) A first aspect of the present disclosure is a toilet roll comprising toilet paper,
wherein the toilet paper is formed by laminating two-ply or three-ply sheets, which have been subjected to embossing and bonded with water and/or glue, and wound into a roll shape,
the roll has a softness of wound roll of not less than 0.5 mm and not more than 4.8 mm, and a wound diameter of not less than 120 mm and not more than 150 mm,
the toilet paper contains a temporary wet strength agent, and a basis weight per ply of not less than 16 g/m² and not more than 26 g/m²,
when a sample of the toilet paper installed on a sample stage is measured using a tissue softness analyzer (TSA) by pressing a blade-equipped rotor from above with a pressing force of 100 mN and then rotating at a rotation speed of 2.0 (/sec), and a vibration of the sample stage is measured by a vibration sensor, an intensity (TS750) of the first spectral maximum peak from the low-frequency side, automatically obtained by software on the TSA, is such that a TS750 (a) on the roll outer surface side is not less than 15 dBV²rms and not more than 50 dBV²rms, a TS750 (b) on the roll inner surface side is not less than 15 dBV²rms and not more than 85 dBV²rms, and a ratio (TS750 (b)/TS750 (a)) is not less than 0.8 and not more than 1.7,
the embossing is provided on at least the ply on the roll outer surface side and includes a first emboss that is linear, and
an area ratio of the first emboss in the toilet paper is not less than 35% and not more than 93%.

(2) A second aspect of the present disclosure is the toilet roll according to (1), wherein a depth of the first emboss in the ply on the roll outer surface side is not less than 0.07 mm and not more than 0.45 mm.

(3) A third aspect of the present disclosure is the toilet roll according to (1), wherein the embossing further includes a dot-shaped second emboss, and a depth of the second emboss in the ply on the roll outer surface side is not less than 0.05 mm and not more than 0.24 mm.

(4) A fourth aspect of the present disclosure is the toilet roll according to (1), wherein a roll wound length is not less than 33 m and not more than 57 m.

(5) A fifth aspect of the present disclosure is the toilet roll according to (1), wherein a roll density is not less than 0.10 g/cm³ and not more than 0.20 g/cm³.

(6) A sixth aspect of the present disclosure is the toilet roll according to (1), wherein a result of a disintegration test as defined in JIS P 4501 for the toilet paper is not less than 25 seconds and not more than 100 seconds.

(7) A seventh aspect of the present disclosure is the toilet roll according to (1), wherein a content of the temporary wet strength agent is not less than 0.005% and not more than 0.2%.

(8) An eighth aspect of the present disclosure is the toilet roll according to (1), wherein the embossing is double embossing provided to the ply on the roll outer surface side and the ply on the roll inner surface side before lamination, and a number of embosses per 1 cm² in the ply on the roll inner surface side is not less than 25 and not more than 95.

(9) A ninth aspect of the present disclosure is the toilet roll according to (1), wherein the embossing is double embossing provided to the ply on the roll outer surface side and the ply on the roll inner surface side before lamination, and a depth of the emboss in the ply on the roll inner surface side is not less than 0.03 mm and not more than 0.35 mm.

(10) A tenth aspect of the present disclosure is the toilet roll according to (1), wherein the roll winding density is not less than 0.48 m/cm² and not more than 1.0 m/cm².

(11) An eleventh aspect of the present disclosure is the toilet roll according to (1), wherein an average peel strength of the toilet paper is not less than 0.09 N/76 mm and not more than 0.32 N/76 mm.

### Advantageous Effect

(12) A twelfth aspect of the present disclosure is the toilet roll according to (1), wherein a maximum value of the peel strength of the toilet paper is not less than 0.20 N/76 mm and not more than 0.60 N/76 mm.

(13) A thirteenth aspect of the present disclosure is the toilet roll according to (1), wherein a basis weight of the product plies of the toilet paper is not less than 32 g/m² and not more than 52 g/m².

(14) A fourteenth aspect of the present disclosure is the toilet roll according to (3), wherein a ratio (C)/(D) of the depth (C) of the first emboss to the depth (D) of the second emboss is not less than 1.05 and not more than 8.0.

(15) A fifteenth aspect of the present disclosure is the toilet roll according to (1), wherein a roll weight is not less than 150 g and not more than 270 g.

According to the present disclosure, it is possible to provide a toilet roll in which the toilet paper contains a temporary wet strength agent and is therefore suitable for use with warm-water washing toilet seats, is resistant to tearing, excels in smoothness, aesthetic appearance and sense of volume of the emboss, and further, the roll has an appropriate wound length and wound diameter, and also exhibits favorable softness.

### BRIEF DESCRIPTION OF THE DRAWINGS

In the accompanying drawings:

FIG. 1 is a perspective view illustrating the external appearance of a toilet roll according to one embodiment of the present disclosure.

FIG. 2 is a diagram depicting an example of embossing applied to toilet paper according to one embodiment of the present disclosure.

FIG. 3 is a diagram indicating a height profile on the X-Y plane by a microscope in gradation.

FIG. 4 is a diagram presenting a height profile on the X-Y plane by a microscope in graph form.

FIG. 5 is a diagram demonstrating a method for determining the depth of embossing applied to toilet paper.

### DETAILED DESCRIPTION

Hereinafter, embodiments for carrying out the present disclosure (hereinafter simply referred to “the present embodiment”) will be described in detail. The following present embodiment is an example for explaining the present disclosure and is not intended to limit the present disclosure to the following contents. The present disclosure can be appropriately modified and implemented within the scope of its gist.

In the drawings, the same reference numerals are assigned to identical elements, and redundant descriptions are omitted. Unless otherwise specified, positional relationships such as up, down, left, and right are based on the positional relationships depicted in the drawings. Furthermore, the dimensional ratios in the drawings are not limited to the illustrated ratios.

### (Toilet Roll)

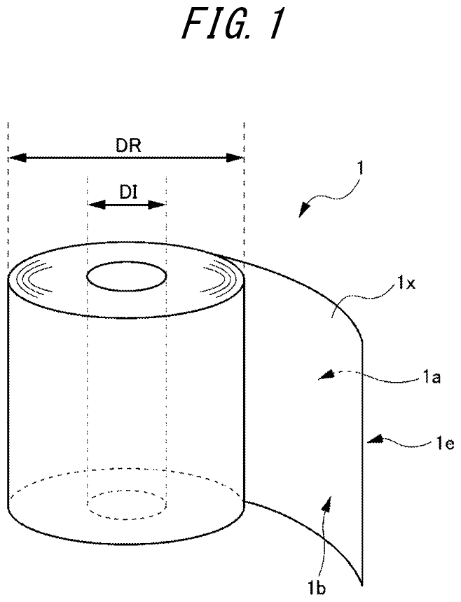

FIG. 1 is a perspective view illustrating the external appearance of a toilet roll 1 according to one embodiment of the present disclosure. The toilet roll 1 according to one embodiment of the present disclosure is a toilet roll 1 in which toilet paper 1x, formed by laminating two-ply or three-ply sheets that have been provided with embossment (embosses) 10 and bonded with water and/or glue (hereinafter also referred to as ply bond glue), is wound into a roll shape. It is preferable that the toilet paper 1x (hereinafter also simply referred to as “sheet”) is provided with perforations in the width direction at approximately regular intervals in the flow direction (not illustrated).

Further, as illustrated in FIG. 1, among the surfaces of the toilet paper 1x, the surface oriented toward the outside of the roll is referred to as the roll outer surface side 1a (the surface of the toilet paper 1x), the surface oriented toward the center of the roll is referred to as the roll inner surface side 1b (the inner surface side of the toilet paper 1x), and the outermost end edge of the wound toilet paper 1x is referred to as the end edge 1e of the outermost winding of the toilet paper 1x.

### (Wound Length and Wound Diameter)

The wound length of the toilet roll 1 is preferably not less than 33 m and not more than 57 m. If the wound length is less than 33 m, the wound length is too short, and as a result, the wound diameter DR described later becomes too small. If the wound length exceeds 57 m, the wound diameter DR described later becomes too large. More preferably, the wound length of the toilet roll 1 is not less than 35 m and not more than 52 m, and even more preferably, not less than 38 m and not more than 46 m.

Further, the wound diameter DR of the toilet roll 1 is not less than 120 mm and not more than 150 mm. If the wound diameter DR is less than 120 mm, the wound diameter DR is too small, and as a result, the wound length becomes too short. If the wound diameter DR exceeds 150 mm, the wound diameter DR becomes too large. Preferably, the wound diameter DR of the toilet roll 1 is not less than 123 mm and not more than 145 mm, and more preferably, not less than 125 mm and not more than 137 mm.

The wound length is measured by actually measuring the length of 10 sheets of the toilet paper 1x between perforations of the toilet roll 1. Then, the number of sheets of the toilet paper 1x in the toilet roll 1 is actually measured, and the wound length is calculated proportionally from the length of 10 sheets and the number of sheets of the toilet paper 1x. For example, if the length of 10 sheets is 1.80 m and the number of sheets of the toilet paper 1x is 200, then 1.80 m×(200/10)=36 m. If the toilet roll 1 does not have perforations, the wound length can be measured directly.

The wound diameter DR of the roll is measured using “a diameter rule” manufactured by Muratec KDS Co., Ltd. For the measurement of the wound length and the wound diameter DR of the roll, four toilet rolls 1 are measured, and the average of the measurement results is used.

### (Softness of Wound Roll)

The softness of wound roll of toilet roll 1 is not less than 0.5 mm and not more than 4.8 mm. If the softness of wound roll is less than 0.5 mm, the softness as a roll product is inferior, and a sense of luxury is difficult to perceive. If the softness of wound roll exceeds 4.8 mm, the roll product becomes too soft and is difficult to use. Preferably, the softness of wound roll of toilet roll 1 is not less than 0.9 mm and not more than 3.5 mm, and more preferably, not less than 1.3 mm and not more than 2.8 mm.

The softness of wound roll of toilet roll 1 is measured as follows using a compression tester (Handy Compression Tester KES-G5 manufactured by Kato Tech Co., Ltd.). The direction parallel to the axis of the toilet roll 1 is defined as the height direction, and the direction perpendicular to the tangent of the circumference of the toilet roll 1 is defined as the radial direction.

First, an acrylic pipe is inserted into the core of the toilet roll 1. The wall thickness of the acrylic pipe is set to 2 mm. The length of the acrylic pipe is made 10 mm longer than the roll width of the toilet roll 1 (for example, if the roll width of the toilet roll 1 is 114 mm, the length of the acrylic pipe is set to 124 mm). The outer diameter of the acrylic pipe is made slightly smaller than the inner diameter of the core of the toilet roll 1, and the size is such that, after inserting the acrylic pipe, when the toilet roll 1 is placed so that its axis is vertical, the toilet roll 1 does not move under its own weight. If it is difficult to insert the acrylic pipe into the core and the outer diameter of the acrylic pipe needs to be made slightly smaller, the wall thickness may be slightly shaved with waterproof sandpaper or the like. The weight of the acrylic pipe is about 31 g when the length is 125 mm and the outer diameter is 38 mm. By inserting the acrylic pipe into the core of the toilet roll 1, even in a toilet roll 1 with a long wound length and high winding density, the softness of wound roll of the toilet roll 1 can be evaluated regardless of the hardness of the core.

Next, the toilet roll 1 is placed horizontally on a hard table so that its axis is horizontal. Then, the head of the compressor (area 2.0 cm²) of the above-mentioned KES-G5 is pressed from above in the radial direction at a speed of 10 mm/min at the center in the height direction of the toilet roll 1. The indentation depth when the pressure applied by the head of the compressor to the roll is 0.5 gf/cm² is defined as T₀, and the indentation depth when the pressure is 100 gf/cm² is defined as Tₘ; the softness of wound roll is defined as (Tₘ−T₀). The position where the above the head of the compressor is pressed into the toilet roll 1 is the center in the height direction, excluding both ends in the height direction. The measurement is performed once for each of four toilet rolls 1, and the average value is used.

### (Sheet Length and Sheet Width)

Furthermore, the length between perforations (sheet length of the toilet paper 1x) is preferably not less than 80 mm and not more than 250 mm, more preferably not less than 88 mm and not more than 160 mm, and even more preferably not less than 95 mm and not more than 120 mm. Furthermore, the sheet width of the toilet paper 1x is preferably not less than 100 mm and not more than 123 mm, more preferably not less than 105 mm and not more than 116 mm, and even more preferably not less than 108 mm and not more than 114 mm. By setting the sheet length and sheet width within the above numerical ranges, when using the toilet roll 1 as in the present application, the area used becomes appropriate, making it suitable for use with warm-water washing toilet seats.

### (Roll Weight)

The roll weight of the toilet roll 1 is preferably not less than 150 g and not more than 270 g. If the roll weight is less than 150 g, the wound diameter DR becomes too small or the wound length becomes too short as a result. If the roll weight exceeds 270 g, the wound diameter DR becomes too large as a result.

Preferably, the roll weight of the toilet roll 1 is not less than 160 g and not more than 240 g, and even more preferably, not less than 170 g and not more than 210 g. The roll weight in the above numerical range refers to the weight per roll per roll width of 114 mm excluding the paper tube (core) in the toilet roll 1; if the roll width is not 114 mm, the weight per 114 mm is calculated proportionally.

### (Outer Diameter of Paper Tube)

The outer diameter DI of the core (paper tube) of the toilet roll 1 of the present disclosure is preferably not less than 25 mm and not more than 48 mm. If the outer diameter DI of the paper tube is less than 25 mm, it becomes difficult to use as a roll product. If the outer diameter DI of the paper tube exceeds 48 mm, the wound diameter DR becomes too large as a result.

Preferably, the outer diameter DI of the paper tube is not less than 35 mm and not more than 46 mm, and even more preferably, not less than 37 mm and not more than 43 mm. The outer diameter DI of the paper tube is measured using “a diameter rule” manufactured by Muratec KDS Co., Ltd. Four paper tubes of the toilet roll 1 are measured, and the average of the measurement results is used.

### (Roll Density)

The roll density of the toilet roll 1 is preferably not less than 0.10 g/cm³ and not more than 0.20 g/cm³. If the roll density is less than 0.10 g/cm³, the toilet roll 1 becomes too soft and is difficult to use. If the roll density exceeds 0.20 g/cm³, the toilet roll 1 is inferior in softness and a sense of luxury is difficult to perceive.

Preferably, the roll density of the toilet roll 1 is not less than 0.11 g/cm³ and not more than 0.18 g/cm³, and even more preferably, not less than 0.12 g/cm³ and not more than 0.15 g/cm³.

The roll density is represented by (roll weight)÷(roll volume). The roll weight is the weight of the toilet roll 1 per roll width of 114 mm. The roll volume is represented by [{cross-sectional area of the roll outer diameter (wound diameter DR)}−(cross-sectional area of the paper tube outer diameter DI)]×roll width (converted to per 114 mm). For example, if the roll weight per roll width of 114 mm (excluding the paper tube) is 162 g, the wound diameter DR is 127 mm, and the paper tube outer diameter DI is 39 mm, the roll density=0.124 g/cm³. If the toilet roll 1 does not have a paper tube, the diameter of the center hole is used as the paper tube outer diameter DI.

### (Winding Density)

Furthermore, the winding density of the toilet roll 1 is preferably not less than 0.48 m/cm² and not more than 1.0 m/cm². If the winding density is less than 0.48 m/cm², the toilet roll 1 becomes too soft and is difficult to use. If the winding density exceeds 1.0 m/cm², the softness as a roll product is inferior, and a sense of luxury is difficult to perceive.

Preferably, the winding density of the toilet roll 1 is not less than 0.50 m/cm² and not more than 0.90 m/cm², and more preferably, not less than 0.60 m/cm² and not more than 0.80 m/cm².

The winding density is represented by (wound length×number of plies)÷(cross-sectional area of the roll). The cross-sectional area of the roll is represented by {cross-sectional area of the roll outer diameter (wound diameter DR)−cross-sectional area of the paper tube 10 outer diameter DI}. For example, if the wound length is 39 m, 2 plies, wound diameter DR is 127 mm, and paper tube outer diameter DI is 39 mm, the winding density=(39 m×2)÷{3.14×(127 mm÷2÷10)²−3.14×(39 mm÷2÷10)²}=0.68 m/cm².

### (Toilet Paper)

The base paper used for the toilet paper 1x according to one embodiment of the present disclosure may be composed of 100% by weight of wood pulp, and may include waste paper pulp, non-wood pulp, deinked pulp, waste paper from liquid beverage paper packs, etc. However, from the viewpoint of quality, the content of pulp other than wood pulp or waste paper is preferably not more than 50% by weight, more preferably not more than 30% by weight, even more preferably not more than 10% by weight, and most preferably 0% by weight.

To obtain the target quality, the content of NBKP (Bleached softwood kraft pulp) is preferably not less than 5% by weight and not more than 55% by weight, more preferably not less than 15% by weight and not more than 45% by weight, and even more preferably not less than 20% by weight and not more than 35% by weight. Further, the content of LBKP (Bleached hardwood kraft pulp) is preferably not less than 45% by weight and not more than 95% by weight, more preferably not less than 55% by weight and not more than 85% by weight, and even more preferably not less than 65% by weight and not more than 80% by weight.

Further, in order for the toilet paper 1x according to one embodiment of the present disclosure to be suitable for use with warm-water washing toilet seats, it contains a temporary wet strength agent. In this case, the content of the temporary wet strength agent is preferably not less than 0.005% and not more than 0.2%. If the content is less than 0.005%, the toilet paper 1x becomes prone to tearing when used with warm-water washing toilet seats. If the content exceeds 0.2%, the toilet paper 1x is inferior in softness.

Preferably, the content of the temporary wet strength agent is not less than 0.01% and not more than 0.1%, and even more preferably, not less than 0.02% and not more than 0.07%. The content of the temporary wet strength agent in this case refers to the weight percentage of the temporary wet strength agent (converted to solid content (active ingredient)) relative to the pulp fiber (in bone dry condition).

In addition to the temporary wet strength agent, additives such as softeners, bulking agents, dyes, dispersants, dry strength agents, drainage aids, pitch control agents, and absorbency enhancers may be used as additives to the stock, depending on the required quality of the final product.

Furthermore, the ply bond glue used in the manufacture of the toilet paper 1x and the toilet roll 1 may be any known glue. Among them, a cellulose-based composition (such as carboxymethyl cellulose) is preferred for the ply bond glue.

Further, the active ingredient (non-volatile content) of the ply bond glue is preferably not less than 0.1% and not more than 5%, more preferably not less than 0.5% and not more than 4%, and even more preferably not less than 1% and not more than 3%. By setting the composition and active ingredient of the ply bond glue as described above, it is possible to obtain a toilet paper 1x that is suitable for use with warm-water washing toilet seats, resistant to tearing, and excellent in smoothness, aesthetic appearance of embosses, and sense of volume, and also to obtain a toilet roll 1 having an appropriate wound length and wound diameter DR and favorable softness of wound roll.

Alternatively, water may be used instead of ply bond glue. By using water, the tactile sensation of the toilet paper 1x can be improved. When water is used, the adhesion between plies may become weak, but as in the toilet roll 1 of the present disclosure, by using the first emboss 11 that is linear described later and setting the emboss area ratio within a certain range, the adhesion between plies can be improved.

It is also possible to use a mixture of ply bond glue and water.

### (Basis Weight)

The basis weight per ply of the toilet paper 1x according to one embodiment of the present disclosure is not less than 16 g/m² and not more than 26 g/m². If the basis weight per ply of the toilet paper 1x is less than 16 g/m², the toilet paper 1x is inferior in sense of volume. If the basis weight per ply of the toilet paper 1x exceeds 26 g/m², the toilet paper 1x is inferior in softness.

Preferably, the basis weight per ply of the toilet paper 1x is not less than 17 g/m² and not more than 24 g/m², and more preferably, not less than 18 g/m² and not more than 22 g/m².

Further, the basis weight of the product ply of the toilet paper 1x is not less than 32 g/m² and not more than 52 g/m². If the basis weight of the product ply of the toilet paper 1x is less than 32 g/m², the toilet paper 1x is inferior in sense of volume. If the basis weight of the product ply of the toilet paper 1x exceeds 52 g/m², the toilet paper 1x is inferior in softness.

Preferably, the basis weight of the product ply of the toilet paper 1x is not less than 34 g/m² and not more than 48 g/m², and more preferably, not less than 36 g/m² and not more than 44 g/m².

The basis weight of the toilet paper 1x is measured in accordance with JIS P 8124.

### (Paper Thickness)

The paper thickness of 10 plies of the toilet paper 1x according to one embodiment of the present disclosure is preferably not less than 1.0 mm and not more than 1.8 mm. If the paper thickness is less than 1.0 mm, the toilet paper 1x is inferior in sense of volume. If the paper thickness exceeds 1.8 mm, the toilet paper 1x is inferior in softness. Preferably, the paper thickness of 10 plies of the toilet paper 1x is not less than 1.1 mm and not more than 1.6 mm, and even more preferably, not less than 1.2 mm and not more than 1.5 mm.

The paper thickness is measured using a thickness gauge (“PEACOCK” dial thickness gauge manufactured by Ozaki Mfg. Co., Ltd.). The measurement conditions are a measurement load of 3.7 kPa and a measurement head diameter of 30 mm; the sample is placed between the measurement head and the measurement table, and the gauge is read when the measurement head is lowered at a speed of not more than 1 mm per second. Two-ply toilet paper 1x is stacked in five sets and measured as 10 sheets, and the result is calculated as the paper thickness of 10 plies. The measurement is repeated 10 times (measuring 10 different locations, not the same location 10 times), and the average of the measurement results is used as the final paper thickness. In the case of three-ply toilet paper 1x, three sets are stacked and measured as nine sheets, and the paper thickness of 10 plies is calculated proportionally.

### (Specific Volume)

The specific volume of the toilet paper 1x according to one embodiment of the present disclosure is preferably not less than 5.0 cm³/g and not more than 9.5 cm³/g. If the specific volume is less than 5.0 cm³/g, the toilet paper 1x is inferior in sense of volume. If the specific volume exceeds 9.5 cm³/g, the toilet paper 1x is inferior in softness.

Preferably, the specific volume of the toilet paper 1x is not less than 5.5 cm³/g and not more than 8.5 cm³/g, and even more preferably, not less than 6.0 cm³/g and not more than 7.5 cm³/g. The specific volume is expressed as the paper thickness of the toilet paper 1x divided by the basis weight, in units of cm³ per g.

### (DMDT and DCDT)

The dry machine direction tensile strength (DMDT) of the toilet paper 1x according to one embodiment of the present disclosure, based on JIS P 8113, is preferably not less than 3.0 N/25 mm and not more than 5.5 N/25 mm. The dry cross direction tensile strength (DCDT) is preferably not less than 1.0 N/25 mm and not more than 2.0 N/25 mm. If the DMDT is less than 3.0 N/25 mm or the DCDT is less than 1.0 N/25 mm, the toilet paper 1x becomes prone to tearing when used with warm-water washing toilet seats. If the DMDT exceeds 5.5 N/25 mm or the DCDT exceeds 2.0 N/25 mm, the toilet paper 1x is inferior in softness.

Preferably, the DMDT is not less than 3.5 N/25 mm and not more than 5.1 N/25 mm, and even more preferably, not less than 3.7 N/25 mm and not more than 4.8 N/25 mm. Preferably, the DCDT is not less than 1.1 N/25 mm and not more than 1.8 N/25 mm, and even more preferably, not less than 1.2 N/25 mm and not more than 1.6 N/25 mm.

In this case, the MD direction refers to the manufacturing direction (Machine Direction, also referred to as the flow direction) during the manufacture of the toilet paper 1x. The CD direction refers to the direction perpendicular to the MD direction (Cross Direction, also referred to as the width direction). The DMDT and DCDT of the toilet paper 1x are measured in accordance with JIS P 8113, using the product ply (two-ply or three-ply) as is.

### (Disintegration Property)

Further, according to one embodiment of the present disclosure, it is preferable that the result of the disintegration test as defined in JIS P 4501 for the toilet paper 1x is not less than 25 seconds and not more than 100 seconds. If the test result is less than 25 seconds, the toilet paper 1x becomes prone to tearing when used with warm-water washing toilet seats. If the test result exceeds 100 seconds, the toilet paper 1x is inferior in softness.

It is more preferable that the result of the disintegration test as defined in JIS P 4501 for the toilet paper 1x is not less than 30 seconds and not more than 80 seconds, and even more preferable that it is not less than 35 seconds and not more than 60 seconds. The disintegration test is conducted in accordance with JIS P 4501, but the toilet paper 1x is separated into a single ply from the state in which it is laminated as two-ply or three-ply. When the product ply is two-ply, the roll outer surface side 1a and the roll inner surface side 1b of the single-ply toilet paper 1x are each measured three times (a total of six times), and the average of the six measurements is taken. When the product ply is three-ply, the roll outer surface side 1a, the roll inner surface side 1b, and the intermediate between the roll outer surface side and the roll inner surface side are each measured twice (a total of six times), and the average of the six measurements is taken. The sample size is set to 114 mm in the width direction×114 mm in the flow direction, but if the roll width is less than 114 mm, the flow direction can be increased (lengthened) so that the sample area is the same. The test piece is sampled so as not to include perforations, but if the interval between perforations is narrow (small) and it is not possible to sample without including perforations, perforations can be included.

### (Peel Strength)

Furthermore, according to one embodiment of the present disclosure, it is preferable that the average peel strength of the toilet paper 1x is not less than 0.09 N/76 mm and not more than 0.32 N/76 mm. If the average peel strength is less than 0.09 N/76 mm, the emboss pattern is light (weak), so the toilet paper 1x is excessively smooth and, conversely, inferior in smoothness. If the average peel strength exceeds 0.32 N/76 mm, the emboss pattern is dense (strong), so the toilet paper 1x is inferior in smoothness.

It is more preferable that the average peel strength of the toilet paper 1x is not less than 0.12 N/76 mm and not more than 0.25 N/76 mm, and even more preferable that it is not less than 0.15 N/76 mm and not more than 0.22 N/76 mm.

Further, according to one embodiment of the present disclosure, it is preferable that the maximum value of the peel strength of the toilet paper 1x is not less than 0.20 N/76 mm and not more than 0.60 N/76 mm. If the maximum value of the peel strength is less than 0.20 N/76 mm, the emboss pattern is light (weak), so the toilet paper 1x is excessively smooth and, conversely, inferior in smoothness. If the maximum value of the peel strength exceeds 0.60 N/76 mm, the emboss pattern is dense (strong), so the toilet paper 1x is inferior in smoothness.

It is more preferable that the maximum value of the peel strength of the toilet paper 1x is not less than 0.27 N/76 mm and not more than 0.50 N/76 mm, and even more preferable that it is not less than 0.33 N/76 mm and not more than 0.45 N/76 mm.

In this case, the peel strength refers to the peel strength between plies of the toilet paper 1x, and the measurement is conducted in accordance with the tensile test method of JIS P 8113, using a tensile tester (“STB-1225S”, manufactured by A&D Company, Limited) under the standard conditions specified in JIS P 8111.

First, a test piece of 76 mm in the width direction and 100 mm in the flow direction is sampled from the toilet paper 1x. Next, the plies are separated by 20 mm in the flow direction. One side of the separated ply is fixed by sandwiching 10 mm in the flow direction in the upper grip (width 80 mm). The other ply is also fixed by sandwiching 10 mm in the flow direction in the lower grip (width 80 mm). The pitch (interval) of the grips is set to 10 mm. Next, the sample is pulled (peeled) at a tensile speed of 100 mm/min, and strength data are collected.

The above strength data are acquired every 0.01 seconds, and after acquiring data from a distance of 40 mm to 80 mm (measurement length 40 mm) from the start of measurement, the average of these data is taken as the average peel strength (N/76 mm). The maximum value among the data up to 80 mm from the start of measurement is taken as the maximum value of the peel strength (N/76 mm). If perforations are included in the range from 40 mm to 80 mm from the start of measurement, the measurement length may be shortened to a range not including perforations, such as from 40 mm to 70 mm (measurement length 30 mm) from the start of measurement.

### (Emboss)

In one embodiment of the present disclosure, emboss 10 is imparted to the toilet paper 1x, and the emboss 10 is provided on at least the ply on the roll outer surface side 1a. The emboss 10 may not be provided on the ply on the roll inner surface side 1b, but it is preferable that it is provided on both the ply on the roll outer surface side 1a and the ply on the roll inner surface side 1b. The emboss 10 may be either single embossing or double embossing, but double embossing is particularly preferable. By doing so, it becomes easier to adjust the wound diameter DR, the softness of wound roll, and the TSA (TS750) described later to a predetermined range.

Regarding the plies to which the emboss 10 is imparted, when the toilet paper 1x is two-ply, it is preferable that the embossing is imparted to both the ply on the roll outer surface side 1a and the ply on the roll inner surface side 1b. When the toilet paper 1x is three-ply, any of the following may be used: two plies on the outer surface side 1a+one ply on the inner surface side 1b, one ply on the outer surface side 1a+two plies on the inner surface side 1b, or one ply on the outer surface side 1a+one ply on the inner surface side 1b (the intermediate ply has no emboss). Among these, it is preferable to impart embossing to two plies on the outer surface side 1a+one ply on the inner surface side 1b, as this makes it less likely for the plies to separate.

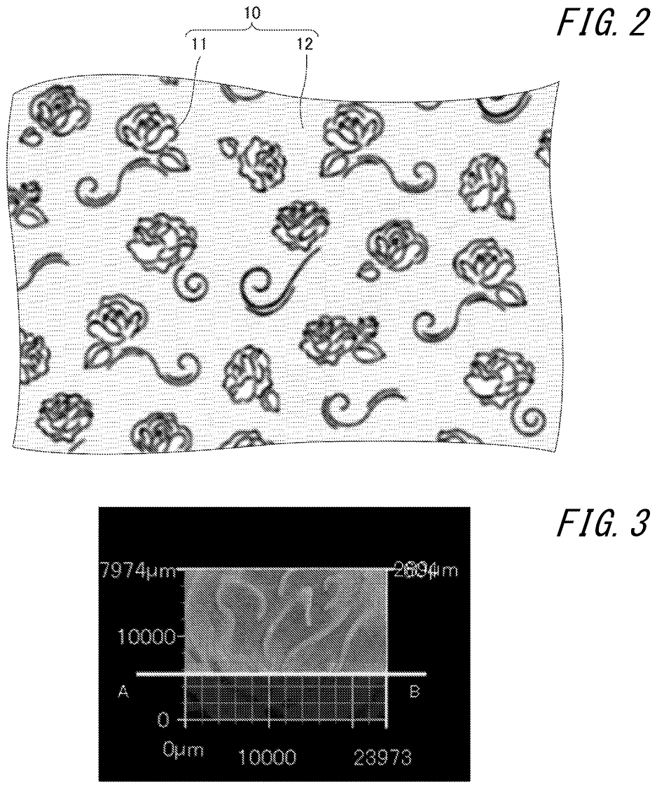

Further, the emboss 10 includes a first emboss 11 that is linear as illustrated in FIG. 2. The shape of the first emboss 11 is preferably a floral pattern as illustrated in FIG. 2, but as long as the area ratio described later is within a predetermined numerical range and the pattern provides an aesthetic appearance, the pattern or design is not particularly limited as long as it is linear.

In the above emboss 10, it is preferable that the depth of the first emboss 11 in the ply on the roll outer surface side 1a is not less than 0.07 mm and not more than 0.45 mm. If the depth of the first emboss 11 is less than 0.07 mm, the aesthetic appearance as embossing is inferior, and the toilet paper 1x is excessively smooth and, conversely, inferior in smoothness. If the depth of the first emboss 11 exceeds 0.45 mm, the toilet paper 1x is inferior in smoothness.

It is more preferable that the depth of the first emboss 11 in the ply on the roll outer surface side 1a is not less than 0.15 mm and not more than 0.40 mm, and even more preferable that it is not less than 0.23 mm and not more than 0.37 mm.

Further, in the emboss 10, the area ratio of the first emboss 11 in the toilet paper 1x is not less than 35% and not more than 93%. If the area ratio is less than 35%, the aesthetic appearance as embossing is inferior, and the toilet paper 1x is excessively smooth and, conversely, inferior in smoothness. If the area ratio exceeds 93%, the aesthetic appearance as embossing is inferior, and the toilet paper 1x is excessively smooth and, conversely, inferior in smoothness. In addition, the toilet roll 1 is inferior in softness as a roll product, and the difference in smoothness between the roll outer surface side 1a and the roll inner surface side 1b of the toilet paper 1x becomes large, resulting in overall inferior smoothness.

It is preferable that the area ratio of the first emboss 11 in the toilet paper 1x is not less than 45% and not more than 85%, and more preferably not less than 55% and not more than 75%.

In addition to the above first emboss 11, it is preferable that the emboss 10 illustrated in FIG. 2 further includes a dot-shaped second emboss 12. By providing the second emboss 12, the tactile sensation (smoothness) of the toilet paper 1x is improved. It is preferable that the second emboss 12 is provided in portions of the toilet paper 1x where the first emboss 11 is not provided (around the first emboss 11), but there may be regions around the first emboss 11 where the second emboss 12 is not provided. The definition of the first emboss 11 that is linear is that the length of the emboss pattern is not less than 3 mm. That is, the first emboss 11 includes those with an emboss pattern length of not less than 3 mm. On the other hand, the second emboss 12 includes those with an emboss pattern length of less than 3 mm.

Further, in the second emboss 12, it is preferable that the depth of the second emboss 12 in the ply on the roll outer surface side 1a is not less than 0.05 mm and not more than 0.24 mm. If the depth of the second emboss 12 is less than 0.05 mm, the aesthetic appearance as embossing is inferior, and the toilet paper 1x is excessively smooth and, conversely, inferior in smoothness. If the depth of the second emboss 12 exceeds 0.24 mm, the toilet paper 1x is inferior in smoothness.

It is more preferable that the depth of the second emboss 12 in the ply on the roll outer surface side 1a is not less than 0.10 mm and not more than 0.21 mm, and even more preferable that it is not less than 0.13 mm and not more than 0.19 mm.

Furthermore, it is preferable that the difference between the depth of the first emboss 11 and the depth of the second emboss 12 in the ply on the roll outer surface side 1a is not less than 0.02 mm and not more than 0.26 mm. If the difference in depth is less than 0.02 mm, the aesthetic appearance as embossing is inferior. If the difference in depth exceeds 0.26 mm, the toilet paper 1x is inferior in smoothness.

It is more preferable that the difference between the depth of the first emboss 11 and the depth of the second emboss 12 in the ply on the roll outer surface side 1a is not less than 0.05 mm and not more than 0.23 mm, and even more preferable that it is not less than 0.10 mm and not more than 0.20 mm.

Further, it is preferable that the ratio (C)/(D) of the depth (C) of the first emboss 11 to the depth (D) of the second emboss 12 is not less than 1.05 and not more than 8.0. If the ratio is less than 1.05, the toilet paper 1x becomes prone to ply separation. If the ratio exceeds 8.0, the toilet paper 1x is inferior in tactile sensation.

It is preferable that the ratio (C)/(D) of the depth (C) of the first emboss 11 to the depth (D) of the second emboss 12 is not less than 1.2 and not more than 4.5, and more preferably not less than 1.5 and not more than 2.5.

When the emboss 10 is double emboss, it is preferable that the number of embosses 10 per 1 cm² in the ply on the roll inner surface side 1b is not less than 25 and not more than 95. If the number of embosses 10 per 1 cm² in the ply on the roll inner surface side 1b is less than 25 or more than 95, the toilet paper 1x is inferior in smoothness or is excessively smooth and, conversely, inferior in smoothness. It is more preferable that the number of embosses 10 per 1 cm² in the ply on the roll inner surface side 1b is not less than 40 and not more than 80, and even more preferable that it is not less than 50 and not more than 70.

Similarly, when the emboss 10 is double embossing, it is preferable that the depth of the emboss 10 in the ply on the roll inner surface side 1b is not less than 0.03 mm and not more than 0.35 mm. If the depth of the emboss 10 in the ply on the roll inner surface side 1b is less than 0.03 mm or more than 0.35 mm, the toilet paper 1x is inferior in smoothness or is excessively smooth and, conversely, inferior in smoothness. It is more preferable that the depth of the emboss 10 in the ply on the roll inner surface side 1b is not less than 0.07 mm and not more than 0.28 mm, and even more preferable that it is not less than 0.10 mm and not more than 0.20 mm.

The depths of the emboss 10, the first emboss 11, and the second emboss 12 are determined by measuring the height difference of the emboss using a microscope. As the microscope, a product named “3D Optical Profilometer VR-3100” manufactured by KEYENCE Corporation can be used. As the image observation, measurement, and image analysis software for the microscope, a product named “VR-HIA” can be used. The measurement conditions are a magnification of 12x and a field of view area of 24 mm×18 mm. The measurement magnification and field of view area may be appropriately changed depending on the size of the emboss 10.

FIG. 3 presents a height profile on the X-Y plane using a microscope, where the height of the surface of the toilet paper 1x is represented by shading. The linear portions in FIG. 3, where the shading differs from the surroundings, indicate individual embosses 10. The depth of the emboss is determined by measuring the height difference of the emboss 10 using the above microscope. The measurement is performed on the roll outer surface side 1a of the toilet paper 1x. At the time of measurement, five windings of the toilet paper 1x are removed from the toilet roll 1, and the sixth winding of the toilet paper 1x is used for measurement in the state of the sheet without changing the number of plies. If there are perforations, all measurements are performed avoiding them.

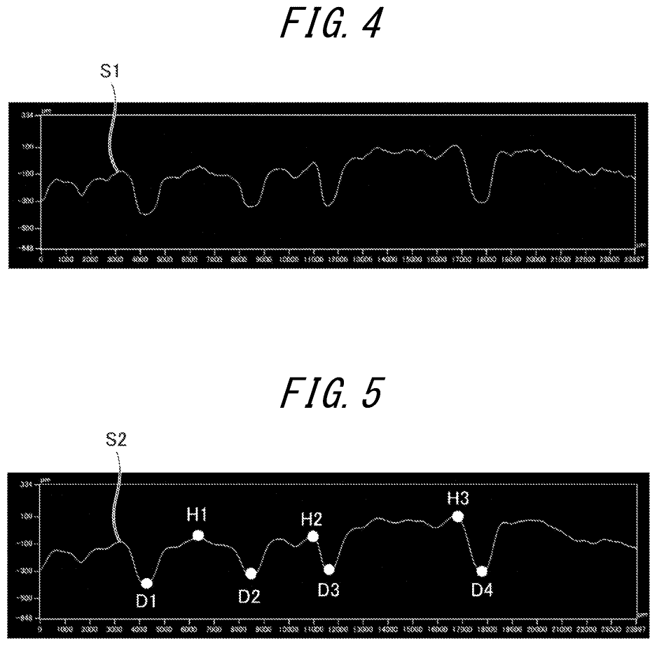

First, as in FIG. 3, a line segment AB is drawn to obtain the height profile of FIG. 4. The line segment AB should be drawn so as to cross the emboss. The line segment AB is drawn in the roll width direction (CD direction) of the toilet paper 1x, but if the interval between embosses is, for example, 2 mm or less and the height of the convex portion described below becomes low, the line segment may be drawn in an oblique direction or in the roll winding direction.

The height profile is a (measured) cross-sectional curve S1 representing the actual surface irregularities of the sample surface of the toilet paper 1x, but it also includes noise (such as fiber clumps on the surface of the toilet paper 1x, fibers extending in a whisker-like manner, or steep peaks caused by areas without fibers), and it is necessary to remove such noise peaks when calculating the height difference of the irregularities.

Therefore, the (measured) cross-sectional curve S1 in FIG. 4 is smoothed using an average weight filter with a filter size of ±12, to obtain the smoothed cross-sectional curve S2 in FIG. 5. The smoothing using the weighted average radio button filter can be automatically obtained by using the above analysis software. In the graph presented in FIG. 5, the concave portions (local minimum) D1 and the adjacent concave portions (local minimum) D2 of the emboss are identified. Next, the convex portion (maximum or local maximum) H1 between D1 and D2 is identified. Similarly, D3, D4, H2, and H3 are identified.

Next, the value of D2 is subtracted from the average value of the vertical axis of H1 and H2, and this is taken as the emboss depth. Similarly, the value of D3 is subtracted from the average value of the vertical axis of H2 and H3, and this is taken as the emboss depth. At this stage, two data points are obtained.

Thereafter, the above measurement is performed at each of three positions obtained by rotating the toilet roll 1 by 90 degrees at a time in the roll winding direction (a total of four measurement positions in the roll winding direction), and the average value of a total of eight locations (2×4) is finally adopted as the emboss depth.

The above measurement is performed for the first emboss 11 and the second emboss 12 on the roll outer surface side 1a, and for the emboss 10 on the roll inner surface side 1b.

As for the method of determining the area ratio of the first emboss 11, first, a 10 cm×10 cm region on the roll outer surface side 1a of the toilet paper 1x is divided into 1 cm×1 cm regions (that is, a total of 100 regions).

Then, among the 1 cm×1 cm regions, the number of regions where the first emboss 11 is not present is counted, and the number obtained by subtracting the number of regions where the first emboss 11 is not present from the total of 100 regions is calculated as the area ratio (%) of the first emboss 11.

### (Measurement Value by TSA)

In the toilet roll 1 according to one embodiment of the present disclosure, when a sample of the toilet paper 1x installed on a sample stage is measured using a tissue softness analyzer (TSA), a blade-equipped rotor is pressed from above with a pressing force of 100 mN, then rotated at a rotation speed of 2.0 (/sec), and the vibration of the sample stage is measured by a vibration sensor, the intensity (TS750) of the first spectral maximum peak from the low-frequency side, automatically obtained by software on the TSA, is such that the TS750 (a) on the roll outer surface side 1a is not less than 15 dBV²rms and not more than 50 dBV²rms. If TS750 (a) is less than 15 dBV²rms, the toilet paper 1x is excessively smooth and, conversely, inferior in smoothness. If TS750 (a) exceeds 50 dBV²rms, the toilet paper 1x is inferior in smoothness.

It is preferable that TS750 (a) is not less than 20 dBV²rms and not more than 45 dBV²rms, and more preferably not less than 25 dBV²rms and not more than 38 dBV²rms.

Further, in the same TS750, the TS750 (b) on the roll inner surface side 1b is not less than 15 dBV²rms and not more than 85 dBV²rms. If TS750 (b) is less than 15 dBV²rms, the toilet paper 1x is excessively smooth and, conversely, inferior in smoothness. If TS750 (b) exceeds 85 dBV²rms, the toilet paper 1x is inferior in smoothness.

It is preferable that TS750 (b) is not less than 20 dBV²rms and not more than 65 dBV²rms, and more preferably not less than 30 dBV²rms and not more than 45 dBV²rms.

Furthermore, in the same TS750, the ratio (TS750 (b)/TS750 (a)) of TS750 (a) on the roll outer surface side 1a to TS750 (b) on the roll inner surface side 1b is not less than 0.8 and not more than 1.7. If the ratio is less than 0.8 or exceeds 1.7, the difference in smoothness between the roll outer surface side 1a and the roll inner surface side 1b of the toilet paper 1x becomes large, resulting in overall inferior smoothness.

It is preferable that the ratio (TS750 (b)/TS750 (a)) is not less than 0.9 and not more than 1.5, and more preferably not less than 1.0 and not more than 1.1.

The above TS750 values are the respective values measured for the portion corresponding to the roll outer surface side 1a or the roll inner surface side 1b of the toilet paper 1x of two-ply or three-ply toilet paper, using a tissue softness analyzer TSA (manufactured by emtec; Tissue Softness Analyzer), with the toilet paper 1x measured as two-ply or three-ply. The lower the TS750 value, the better the smoothness, but if the TS750 value is too low, the paper becomes excessively smooth and, conversely, inferior in smoothness.

The method for measuring the TS750 value using the tissue softness analyzer TSA and the measuring device used are described in detail, for example, in JP 2013-236904 A. For various measurement methods using the tissue softness analyzer TSA, refer to the above patent literature.

### (Method for Manufacturing Toilet Roll)

Regarding the manufacturing process of the toilet paper 1x and the toilet roll 1, for example, as follows, (1) papermaking and creping, (2) embossing and ply bonding with ply bond glue, and (3) roll winding processing, can be performed in this order.

In the papermaking step (1), it is preferable to use a general dry crepe papermaking machine without using a papermaking machine for bulky paper such as TAD paper.

As described above, according to the present embodiment, since the toilet paper contains a temporary wet strength agent, it is suitable for use with warm-water washing toilet seats, is resistant to tearing, excels in smoothness, aesthetic appearance of embosses, and sense of volume, and further, the roll has an appropriate wound length and wound diameter, and it is possible to provide a toilet roll that also exhibits favorable roll softness.

### Examples

The present disclosure will be described in further detail by way of the following examples and comparative examples; however, the present disclosure is not limited in any way by these examples.

Under the respective conditions indicated in Tables 1 to 3, two-ply toilet paper and toilet rolls of Examples 1 to 29 and Comparative Examples 1 to 10 were produced, and the following evaluations were conducted.

Each evaluation was performed after maintaining the samples under equilibrium conditions at the temperature and humidity specified in JIS P8111 (23±1 °C, 50±2% RH). Further, parameters other than the evaluations described below were measured in accordance with the above-mentioned standards or measurement methods.

(Resistance to Tearing when Used with Warm-Water Washing Toilet Seats)

Thirty monitors evaluated the resistance to tearing of the toilet paper when used with warm-water washing toilet seats. The evaluation was conducted in four grades based on the number of monitors who determined that the toilet paper was resistant to tearing even when used with warm-water washing toilet seats.
4:28 or more monitors determined that the toilet paper was resistant to tearing even when used with warm-water washing toilet seats.
3:25 to 27 monitors determined that the toilet paper was resistant to tearing even when used with warm-water washing toilet seats.
2:21 to 24 monitors determined that the toilet paper was resistant to tearing even when used with warm-water washing toilet seats.
1:20 or fewer monitors determined that the toilet paper was resistant to tearing even when used with warm-water washing toilet seats.

### (Wound Length)

The wound length of the toilet roll was evaluated. The evaluation was conducted in four grades based on the wound length of the toilet roll in each example and comparative example.
4: The wound length of the toilet roll was not less than 38 m and not more than 46 m.
3: The wound length of the toilet roll was not less than 35 m and less than 38 m, or more than 46 m and not more than 52 m.
2: The wound length of the toilet roll was not less than 33 m and less than 35 m, or more than 52 m and not more than 57 m.
1: The wound length of the toilet roll was less than 33 m or more than 57 m.

### (Wound Diameter)

The wound diameter of the toilet roll was evaluated. The evaluation was conducted in four grades based on the wound diameter of the toilet roll in each example and comparative example.
4: The wound diameter of the toilet roll was not less than 125 mm and not more than 137 mm.
3: The wound diameter of the toilet roll was not less than 123 mm and less than 125 mm, or more than 137 mm and not more than 145 mm.
2: The wound diameter of the toilet roll was not less than 120 mm and less than 123 mm, or more than 145 mm and not more than 150 mm.
1: The wound diameter of the toilet roll was less than 120 mm or more than 150 mm.

### (Tactile Sensation (Smoothness))

Thirty monitors evaluated the tactile sensation (smoothness) of the toilet paper. The evaluation was conducted in four grades based on the number of monitors who determined that the smoothness of the toilet paper was favorable.
4:28 or more monitors determined that the smoothness of the toilet paper was favorable.
3:25 to 27 monitors determined that the smoothness of the toilet paper was favorable.
2:21 to 24 monitors determined that the smoothness of the toilet paper was favorable.
1:20 or fewer monitors determined that the smoothness of the toilet paper was favorable.

### (Tactile Sensation (Difference in Smoothness Between Outer Surface Side and Inner Surface Side))

Thirty monitors evaluated the tactile sensation (difference in smoothness between the outer surface side and the inner surface side) of the toilet paper. The evaluation was conducted in four grades based on the number of monitors who determined that the difference in smoothness between the outer surface side and the inner surface side was small and favorable.
4:28 or more monitors determined that the difference in smoothness between the outer surface side and the inner surface side was small and the tactile sensation was favorable.
3:25 to 27 monitors determined that the difference in smoothness between the outer surface side and the inner surface side was small and the tactile sensation was favorable.
2:21 to 24 monitors determined that the difference in smoothness between the outer surface side and the inner surface side was small and the tactile sensation was favorable.
1:20 or fewer monitors determined that the difference in smoothness between the outer surface side and the inner surface side was small and the tactile sensation was favorable.

### (Roll Softness)

Thirty monitors evaluated the roll softness of the toilet roll. The evaluation was conducted in four grades based on the number of monitors who determined that the roll softness was favorable.
4:28 or more monitors determined that the roll softness was favorable.
3:25 to 27 monitors determined that the roll softness was favorable.
2:21 to 24 monitors determined that the roll softness was favorable.
1:20 or fewer monitors determined that the roll softness was favorable.

### (Sense of Volume)

Thirty monitors evaluated the sense of volume of the toilet paper. The evaluation was conducted in four grades based on the number of monitors who determined the sense of volume to be excellent.
4:28 or more monitors determined the sense of volume to be excellent.
3:25 to 27 monitors determined the sense of volume to be excellent.
2:21 to 24 monitors determined the sense of volume to be excellent.
1:20 or fewer monitors determined the sense of volume to be excellent.

### (Softness of Toilet Paper)

Thirty monitors evaluated the softness of the toilet paper. The evaluation was conducted in four grades based on the number of monitors who determined that the toilet paper was soft.
4:28 or more monitors determined that the toilet paper was soft.
3:25 to 27 monitors determined that the toilet paper was soft.
2:21 to 24 monitors determined that the toilet paper was soft.
1:20 or fewer monitors determined that the toilet paper was soft.

### (Aesthetic Appearance of Embosses)

Thirty monitors evaluated the aesthetic appearance of the embosses of the toilet paper. The evaluation was conducted in four grades based on the number of monitors who determined that the aesthetic appearance was excellent.
4:28 or more monitors determined that the aesthetic appearance was excellent.
3:25 to 27 monitors determined that the aesthetic appearance was excellent.
2:21 to 24 monitors determined that the aesthetic appearance was excellent.
1:20 or fewer monitors determined that the aesthetic appearance was excellent.

Tables 1 to 3 present the conditions and evaluation results of each example and each comparative example.

TABLE 1

| | Evaluation | | Exam- | Exam- | Exam- | Exam- | Exam- | Exam- | Exam- | Exam- |
|---|---|---|---|---|---|---|---|---|---|---|
| | items | | ple 1 | ple 2 | ple 3 | ple 4 | ple 5 | ple 6 | ple 7 | ple 8 |
| Toilet | Basis | g/m² | 19.8 | 19.7 | 18.2 | 20.0 | 21.7 | 20.4 | 20.2 | 20.0 |
| paper | weight | (1 ply) | | | | | | | | |
| | Paper | mm | 1.33 | 1.28 | 1.21 | 1.30 | 1.43 | 1.27 | 1.31 | 1.40 |
| | thickness | (10 plies) | | | | | | | | |
| | Specific | cm³/g | 6.7 | 6.5 | 6.6 | 6.5 | 6.6 | 6.2 | 6.5 | 7.0 |
| | volume | | | | | | | | | |
| | DMDT | N/25 mm | 4.11 | 4.07 | 3.72 | 4.10 | 4.53 | 4.07 | 4.15 | 4.10 |
| | | (product ply) | | | | | | | | |
| | DCDT | N/25 mm | 1.34 | 1.31 | 1.24 | 1.33 | 1.45 | 1.35 | 1.34 | 1.35 |
| | | (product ply) | | | | | | | | |
| | Disintegration | seconds | 46 | 42 | 59 | 44 | 36 | 48 | 47 | 44 |
| | property | (1 ply) | | | | | | | | |
| | Content of | % | 0.04 | 0.04 | 0.07 | 0.04 | 0.02 | 0.04 | 0.04 | 0.04 |
| | temporary | | | | | | | | | |
| | wet strength | | | | | | | | | |
| | agent | | | | | | | | | |
| | Depth of | mm | 0.31 | 0.29 | 0.28 | 0.31 | 0.33 | 0.32 | 0.31 | 0.30 |
| | first | | | | | | | | | |
| | emboss | | | | | | | | | |
| | (outer | | | | | | | | | |
| | surface | | | | | | | | | |
| | side): C | | | | | | | | | |
| | Depth of | mm | 0.14 | 0.16 | 0.13 | 0.16 | 0.17 | 0.14 | 0.14 | 0.14 |
| | second | | | | | | | | | |
| | emboss | | | | | | | | | |
| | (outer | | | | | | | | | |
| | surface | | | | | | | | | |
| | side): D | | | | | | | | | |
| | C − D | mm | 0.17 | 0.13 | 0.15 | 0.15 | 0.16 | 0.18 | 0.17 | 0.16 |
| | C/D | — | 2.21 | 1.81 | 2.15 | 1.94 | 1.94 | 2.29 | 2.21 | 2.14 |
| | Area ratio of | % | 66 | 64 | 73 | 66 | 50 | 68 | 66 | 66 |
| | first emboss | | | | | | | | | |
| | (outer | | | | | | | | | |
| | surface side) | | | | | | | | | |
| | Number of | pieces/1 | 61 | 57 | 50 | 56 | 70 | 62 | 61 | 57 |
| | embosses | cm² | | | | | | | | |
| | (inner | | | | | | | | | |
| | surface side) | | | | | | | | | |
| | Emboss depth | mm | 0.14 | 0.15 | 0.11 | 0.15 | 0.19 | 0.15 | 0.16 | 0.18 |
| | (inner | | | | | | | | | |
| | surface side) | | | | | | | | | |
| | TS750 (outer | dBV² | 33.3 | 33.4 | 31.9 | 33.0 | 34.7 | 33.7 | 32.5 | 34.3 |
| | surface side) | rms | | | | | | | | |
| | (a) | | | | | | | | | |
| | TS750 (inner | dBV² | 33.9 | 34.1 | 33.0 | 34.0 | 36.5 | 33.6 | 34.2 | 34.9 |
| | surface side) | rms | | | | | | | | |
| | (b) | | | | | | | | | |
| | (b)/(a) | — | 1.02 | 1.02 | 1.03 | 1.03 | 1.05 | 1.00 | 1.05 | 1.02 |
| | Ply | Glue/ | glue | glue | glue | glue | glue | glue | glue | glue |
| | bonding | water | | | | | | | | |
| | Peel strength | N/76 | 0.20 | 0.18 | 0.20 | 0.18 | 0.17 | 0.20 | 0.19 | 0.18 |
| | (average) | mm | | | | | | | | |
| | Peel strength | N/76 | 0.43 | 0.40 | 0.38 | 0.37 | 0.36 | 0.42 | 0.40 | 0.40 |
| | (maximum) | mm | | | | | | | | |
| Toilet roll | Wound | m | 34 | 37 | 39 | 41 | 45 | 51 | 56 | 33 |
| | length | | | | | | | | | |
| | Roll weight | g | 153 | 166 | 162 | 187 | 223 | 237 | 258 | 150 |
| | (per 114 mm | | | | | | | | | |
| | width) | | | | | | | | | |
| | Wound | mm | 120 | 124 | 127 | 130 | 135 | 144 | 150 | 135 |
| | diameter | | | | | | | | | |
| | Outer | mm | 39 | 39 | 39 | 39 | 39 | 39 | 39 | 39 |
| | diameter of | | | | | | | | | |
| | paper tube | | | | | | | | | |
| | Winding | m/cm² | 0.67 | 0.68 | 0.68 | 0.68 | 0.69 | 0.68 | 0.68 | 0.50 |
| | density | | | | | | | | | |
| | Softness of | mm | 1.9 | 2.0 | 2.6 | 2.0 | 1.4 | 1.9 | 2.0 | 4.5 |
| | wound roll | | | | | | | | | |
| | Roll | g/cm³ | 0.133 | 0.134 | 0.124 | 0.136 | 0.149 | 0.138 | 0.137 | 0.101 |
| | density | | | | | | | | | |

| | | | | | | | | | |
|---|---|---|---|---|---|---|---|---|---|
| Evaluation | Resistance to tearing | 4 | 4 | 4 | 4 | 4 | 4 | 4 | 4 |
| | when used with warm- | | | | | | | | |
| | water washing toilet | | | | | | | | |
| | seats | | | | | | | | |
| | Wound length | 2 | 3 | 4 | 4 | 4 | 3 | 2 | 2 |
| | Wound diameter | 2 | 3 | 4 | 4 | 4 | 3 | 2 | 4 |
| | Ractile sensation | 4 | 4 | 4 | 4 | 4 | 4 | 4 | 4 |
| | (smoothness) | | | | | | | | |
| | Difference in | 4 | 4 | 4 | 4 | 4 | 4 | 4 | 4 |
| | smoothness between | | | | | | | | |
| | outer surface side and | | | | | | | | |
| | inner surface side | | | | | | | | |
| | Roll softness | 4 | 4 | 4 | 4 | 4 | 4 | 4 | 2 |
| | Sense of volume | 4 | 4 | 4 | 4 | 4 | 4 | 4 | 4 |
| | Softness of | 4 | 4 | 4 | 4 | 4 | 4 | 4 | 4 |
| | toilet paper | | | | | | | | |
| | Aesthetic appearance | 4 | 4 | 4 | 4 | 4 | 4 | 4 | 4 |
| | of embosses | | | | | | | | |

| | Evaluation | | Exam- | Exam- | Exam- | Exam- | Exam- | Exam- | Exam- |
|---|---|---|---|---|---|---|---|---|---|
| | items | | ple 9 | ple 10 | ple 11 | ple 12 | ple 13 | ple 14 | ple 15 |
| Toilet | Basis | g/m² | 20.0 | 20.2 | 20.1 | 16.0 | 17.0 | 24.0 | 26.0 |
| paper | weight | (1 ply) | | | | | | | |
| | Paper | mm | 1.36 | 1.26 | 1.22 | 1.02 | 1.11 | 1.53 | 1.72 |
| | thickness | (10 plies) | | | | | | | |
| | Specific | cm³/g | 6.8 | 6.2 | 6.1 | 6.4 | 6.5 | 6.4 | 6.6 |
| | volume | | | | | | | | |
| | DMDT | N/25 mm | 4.11 | 4.09 | 4.10 | 3.06 | 3.62 | 4.94 | 5.35 |
| | | (product ply) | | | | | | | |
| | DCDT | N/25 mm | 1.36 | 1.35 | 1.36 | 1.04 | 1.18 | 1.71 | 1.92 |
| | | (product ply) | | | | | | | |
| | Disintegration | seconds | 45 | 44 | 45 | 26 | 30 | 78 | 97 |
| | property | (1 ply) | | | | | | | |
| | Content of | % | 0.04 | 0.04 | 0.04 | 0.005 | 0.01 | 0.10 | 0.20 |
| | temporary | | | | | | | | |
| | wet strength | | | | | | | | |
| | agent | | | | | | | | |
| | Depth of | mm | 0.29 | 0.29 | 0.31 | 0.26 | 0.27 | 0.34 | 0.37 |
| | first | | | | | | | | |
| | emboss | | | | | | | | |
| | (outer | | | | | | | | |
| | surface | | | | | | | | |
| | side): C | | | | | | | | |
| | Depth of | mm | 0.14 | 0.15 | 0.14 | 0.10 | 0.12 | 0.18 | 0.20 |
| | second | | | | | | | | |
| | emboss | | | | | | | | |
| | (outer | | | | | | | | |
| | surface | | | | | | | | |
| | side): D | | | | | | | | |
| | C − D | mm | 0.15 | 0.14 | 0.17 | 0.16 | 0.15 | 0.16 | 0.17 |
| | C/D | — | 2.07 | 1.93 | 2.21 | 2.60 | 2.25 | 1.89 | 1.85 |
| | Area ratio of | % | 67 | 66 | 65 | 65 | 67 | 66 | 65 |
| | first emboss | | | | | | | | |
| | (outer | | | | | | | | |
| | surface side) | | | | | | | | |
| | Number of | pieces/1 | 56 | 63 | 60 | 58 | 60 | 60 | 62 |
| | embosses | cm² | | | | | | | |
| | (inner | | | | | | | | |
| | surface side) | | | | | | | | |
| | Emboss depth | mm | 0.17 | 0.13 | 0.12 | 0.11 | 0.13 | 0.18 | 0.19 |
| | (inner | | | | | | | | |
| | surface side) | | | | | | | | |
| | TS750 (outer | dBV² | 34.0 | 32.2 | 32.0 | 30.2 | 31.1 | 35.5 | 36.2 |
| | surface side) | rms | | | | | | | |
| | (a) | | | | | | | | |
| | TS750 (inner | dBV² | 34.5 | 32.9 | 32.4 | 31.8 | 32.5 | 36.6 | 37.9 |
| | surface side) | rms | | | | | | | |
| | (b) | | | | | | | | |
| | (b)/(a) | — | 1.01 | 1.02 | 1.01 | 1.05 | 1.05 | 1.03 | 1.05 |
| | Ply | Glue/ | glue | glue | glue | glue | glue | glue | glue |
| | bonding | water | | | | | | | |
| | Peel strength | N/76 | 0.19 | 0.17 | 0.18 | 0.18 | 0.19 | 0.17 | 0.19 |
| | (average) | mm | | | | | | | |
| | Peel strength | N/76 | 0.36 | 0.43 | 0.40 | 0.40 | 0.41 | 0.43 | 0.42 |
| | (maximum) | mm | | | | | | | |
| Toilet roll | Wound | m | 35 | 52 | 57 | 47 | 44 | 38 | 35 |
| | length | | | | | | | | |
| | Roll weight | g | 160 | 239 | 261 | 171 | 171 | 208 | 207 |
| | (per 114 mm | | | | | | | | |
| | width) | | | | | | | | |
| | Wound | mm | 131 | 129 | 127 | 125 | 125 | 137 | 137 |
| | diameter | | | | | | | | |
| | Outer | mm | 39 | 39 | 39 | 39 | 39 | 39 | 39 |
| | diameter of | | | | | | | | |
| | paper tube | | | | | | | | |
| | Winding | m/cm² | 0.57 | 0.88 | 0.99 | 0.85 | 0.79 | 0.56 | 0.52 |
| | density | | | | | | | | |
| | Softness of | mm | 3.2 | 1.0 | 0.6 | 1.8 | 1.9 | 2.1 | 2.2 |
| | wound roll | | | | | | | | |
| | Roll | g/cm³ | 0.114 | 0.177 | 0.200 | 0.136 | 0.135 | 0.135 | 0.134 |
| | density | | | | | | | | |

| | | | | | | | | |
|---|---|---|---|---|---|---|---|---|
| Evaluation | Resistance to tearing | 4 | 4 | 4 | 2 | 3 | 4 | 4 |
| | when used with warm- | | | | | | | |
| | water washing toilet | | | | | | | |
| | seats | | | | | | | |
| | Wound length | 3 | 3 | 2 | 3 | 4 | 4 | 3 |
| | Wound diameter | 4 | 4 | 4 | 4 | 4 | 4 | 4 |
| | Ractile sensation | 4 | 4 | 4 | 4 | 4 | 4 | 4 |
| | (smoothness) | | | | | | | |
| | Difference in | 4 | 4 | 4 | 4 | 4 | 4 | 4 |
| | smoothness between | | | | | | | |
| | outer surface side and | | | | | | | |
| | inner surface side | | | | | | | |
| | Roll softness | 3 | 3 | 2 | 4 | 4 | 4 | 4 |
| | Sense of volume | 4 | 4 | 4 | 2 | 3 | 3 | 2 |
| | Softness of | 4 | 4 | 4 | 4 | 4 | 3 | 2 |
| | toilet paper | | | | | | | |
| | Aesthetic appearance | 4 | 4 | 4 | 4 | 4 | 4 | 4 |
| | of embosses | | | | | | | |

TABLE 2

| | Evaluation | | Exam- | Exam- | Exam- | Exam- | Exam- | Exam- | Exam- | Exam- |
|---|---|---|---|---|---|---|---|---|---|---|
| | items | | ple 16 | ple 17 | ple 18 | ple 19 | ple 20 | ple 21 | ple 22 | ple 23 |
| Toilet | Basis | g/m² | 20.0 | 20.2 | 20.1 | 20.0 | 20.1 | 19.9 | 20.1 | 20.0 |
| paper | weight | (1 ply) | | | | | | | | |
| | Paper | mm | 1.09 | 1.19 | 1.42 | 1.55 | 1.37 | 1.34 | 1.26 | 1.23 |
| | thickness | (10 plies) | | | | | | | | |
| | Specific | cm³/g | 5.5 | 5.9 | 7.1 | 7.8 | 6.8 | 6.7 | 6.3 | 6.2 |
| | volume | | | | | | | | | |
| | DMDT | N/25 mm | 4.29 | 4.20 | 3.96 | 3.84 | 3.97 | 4.03 | 4.18 | 4.30 |
| | | (product ply) | | | | | | | | |
| | DCDT | N/25 mm | 1.42 | 1.38 | 1.25 | 1.20 | 1.27 | 1.30 | 1.36 | 1.40 |
| | | (product ply) | | | | | | | | |
| | Disintegration | seconds | 45 | 44 | 43 | 43 | 36 | 40 | 50 | 54 |
| | property | (1 ply) | | | | | | | | |
| | Content of | % | 0.04 | 0.04 | 0.04 | 0.04 | 0.04 | 0.04 | 0.04 | 0.04 |
| | temporary | | | | | | | | | |
| | wet strength | | | | | | | | | |
| | agent | | | | | | | | | |
| | Depth of | mm | 0.08 | 0.17 | 0.38 | 0.46 | 0.31 | 0.30 | 0.30 | 0.32 |
| | first | | | | | | | | | |
| | emboss | | | | | | | | | |
| | (outer | | | | | | | | | |
| | surface | | | | | | | | | |
| | side): C | | | | | | | | | |
| | Depth of | mm | 0.06 | 0.12 | 0.20 | 0.22 | 0.16 | 0.15 | 0.17 | 0.16 |
| | second | | | | | | | | | |
| | emboss | | | | | | | | | |
| | (outer | | | | | | | | | |
| | surface | | | | | | | | | |
| | side): D | | | | | | | | | |
| | C − D | mm | 0.02 | 0.05 | 0.18 | 0.24 | 0.15 | 0.15 | 0.13 | 0.16 |
| | C/D | — | 1.33 | 1.42 | 1.90 | 2.09 | 1.94 | 2.00 | 1.76 | 2.00 |
| | Area ratio of | % | 65 | 66 | 65 | 67 | 33 | 44 | 84 | 92 |
| | first emboss | | | | | | | | | |
| | (outer | | | | | | | | | |
| | surface side) | | | | | | | | | |
| | Number of | pieces/1 | 70 | 62 | 60 | 58 | 59 | 58 | 56 | 60 |
| | embosses | cm² | | | | | | | | |
| | (inner | | | | | | | | | |
| | surface side) | | | | | | | | | |
| | Emboss depth | mm | 0.16 | 0.16 | 0.14 | 0.15 | 0.15 | 0.14 | 0.16 | 0.15 |
| | (inner | | | | | | | | | |
| | surface side) | | | | | | | | | |
| | TS750 (outer | dBV² | 18.9 | 22.5 | 40.5 | 46.3 | 16.1 | 20.4 | 44.3 | 49.0 |
| | surface side) | rms | | | | | | | | |
| | (a) | | | | | | | | | |
| | TS750 (inner | dBV² | 18.5 | 23.4 | 46.5 | 56.7 | 15.1 | 20.9 | 64.4 | 81.4 |
| | surface side) | rms | | | | | | | | |
| | (b) | | | | | | | | | |
| | (b)/(a) | — | 0.98 | 1.04 | 1.15 | 1.22 | 0.94 | 1.02 | 1.45 | 1.66 |
| | Ply | Glue/ | glue | glue | glue | glue | glue | glue | glue | glue |
| | bonding | water | | | | | | | | |
| | Peel strength | N/76 | 0.10 | 0.13 | 0.23 | 0.30 | 0.10 | 0.13 | 0.24 | 0.31 |
| | (average) | mm | | | | | | | | |
| | Peel strength | N/76 | 0.22 | 0.29 | 0.46 | 0.57 | 0.34 | 0.35 | 0.40 | 0.42 |
| | (maximum) | mm | | | | | | | | |
| Toilet | Wound | m | 41 | 41 | 41 | 41 | 38 | 39 | 42 | 43 |
| roll | length | | | | | | | | | |
| | Roll weight | g | 187 | 189 | 188 | 187 | 174 | 177 | 192 | 196 |
| | (per 114 mm | | | | | | | | | |
| | width) | | | | | | | | | |
| | Wound | mm | 121 | 126 | 136 | 140 | 130 | 130 | 130 | 130 |
| | diameter | | | | | | | | | |
| | Outer | mm | 39 | 39 | 39 | 39 | 39 | 39 | 39 | 39 |
| | diameter of | | | | | | | | | |
| | paper tube | | | | | | | | | |
| | Winding | m/cm² | 0.80 | 0.73 | 0.62 | 0.58 | 0.63 | 0.65 | 0.70 | 0.71 |
| | density | | | | | | | | | |
| | Softness of | mm | 1.4 | 1.7 | 2.3 | 2.8 | 2.3 | 2.2 | 1.9 | 1.8 |
| | wound roll | | | | | | | | | |
| | Roll | g/cm³ | 0.159 | 0.147 | 0.124 | 0.116 | 0.127 | 0.129 | 0.140 | 0.142 |
| | density | | | | | | | | | |

| | | | | | | | | | |
|---|---|---|---|---|---|---|---|---|---|
| Evaluation | Resistance to tearing | 4 | 4 | 4 | 4 | 4 | 4 | 4 | 4 |
| | when used with warm- | | | | | | | | |
| | water washing toilet | | | | | | | | |
| | seats | | | | | | | | |
| | Wound length | 4 | 4 | 4 | 4 | 4 | 4 | 4 | 4 |
| | Wound diameter | 2 | 4 | 4 | 3 | 4 | 4 | 4 | 4 |
| | Ractile sensation | 2 | 3 | 3 | 2 | 2 | 3 | 3 | 2 |
| | (smoothness) | | | | | | | | |
| | Difference in | 3 | 4 | 3 | 3 | 3 | 4 | 3 | 2 |
| | smoothness between | | | | | | | | |
| | outer surface side and | | | | | | | | |
| | inner surface side | | | | | | | | |
| | Roll softness | 4 | 4 | 4 | 4 | 4 | 4 | 4 | 4 |
| | Sense of volume | 2 | 3 | 4 | 4 | 4 | 4 | 4 | 4 |
| | Softness of | 2 | 3 | 4 | 3 | 4 | 4 | 3 | 2 |
| | toilet paper | | | | | | | | |
| | Aesthetic appearance | 2 | 3 | 4 | 4 | 2 | 3 | 3 | 2 |
| | of embosses | | | | | | | | |

| | Evaluation | | Exam- | Exam- | Exam- | Exam- | Exam- | Exam- |
|---|---|---|---|---|---|---|---|---|
| | items | | ple 24 | ple 25 | ple 26 | ple 27 | ple 28 | ple 29 |
| Toilet | Basis | g/m² | 20.1 | 19.8 | 20.1 | 19.8 | 20.0 | 20.0 |
| paper | weight | (1 ply) | | | | | | |
| | Paper | mm | 1.33 | 1.32 | 1.27 | 1.26 | 1.30 | 1.32 |
| | thickness | (10 plies) | | | | | | |
| | Specific | cm³/g | 6.6 | 6.7 | 6.3 | 6.4 | 6.5 | 6.6 |
| | volume | | | | | | | |
| | DMDT | N/25 mm | 4.02 | 4.06 | 4.15 | 4.19 | 4.11 | 4.07 |
| | | (product ply) | | | | | | |
| | DCDT | N/25 mm | 1.30 | 1.31 | 1.34 | 1.36 | 1.32 | 1.31 |
| | | (product ply) | | | | | | |
| | Disintegration | seconds | 36 | 40 | 50 | 54 | 42 | 40 |
| | property | (1 ply) | | | | | | |
| | Content of | % | 0.04 | 0.04 | 0.04 | 0.04 | 0.04 | 0.04 |
| | temporary | | | | | | | |
| | wet strength | | | | | | | |
| | agent | | | | | | | |
| | Depth of | mm | 0.32 | 0.30 | 0.30 | 0.32 | 0.31 | 0.30 |
| | first | | | | | | | |
| | emboss | | | | | | | |
| | (outer | | | | | | | |
| | surface | | | | | | | |
| | side): C | | | | | | | |
| | Depth of | mm | 0.16 | 0.15 | 0.16 | 0.16 | 0.16 | 0.15 |
| | second | | | | | | | |
| | emboss | | | | | | | |
| | (outer | | | | | | | |
| | surface | | | | | | | |
| | side): D | | | | | | | |
| | C − D | mm | 0.16 | 0.15 | 0.14 | 0.16 | 0.15 | 0.15 |
| | C/D | — | 2.00 | 2.00 | 1.88 | 2.00 | 1.94 | 2.00 |
| | Area ratio of | % | 72 | 69 | 63 | 59 | 66 | 66 |
| | first emboss | | | | | | | |
| | (outer | | | | | | | |
| | surface side) | | | | | | | |
| | Number of | pieces/1 | 59 | 58 | 59 | 60 | 56 | 56 |
| | embosses | cm² | | | | | | |
| | (inner | | | | | | | |
| | surface side) | | | | | | | |
| | Emboss depth | mm | 0.15 | 0.15 | 0.14 | 0.14 | 0.15 | 0.15 |
| | (inner | | | | | | | |
| | surface side) | | | | | | | |
| | TS750 (outer | dBV² | 24.6 | 28.9 | 37.7 | 41.7 | 32.8 | 32.7 |
| | surface side) | rms | | | | | | |
| | (a) | | | | | | | |
| | TS750 (inner | dBV² | 24.9 | 30.2 | 50.5 | 62.5 | 33.9 | 33.9 |
| | surface side) | rms | | | | | | |
| | (b) | | | | | | | |
| | (b)/(a) | — | 1.01 | 1.04 | 1.34 | 1.50 | 1.03 | 1.04 |
| | Ply | Glue/ | glue | glue | glue | glue | mixture of | water |
| | bonding | water | | | | | glue/water | |
| | Peel strength | N/76 | 0.11 | 0.14 | 0.24 | 0.30 | 0.16 | 0.15 |
| | (average) | mm | | | | | | |
| | Peel strength | N/76 | 0.22 | 0.29 | 0.46 | 0.57 | 0.34 | 0.33 |
| | (maximum) | mm | | | | | | |
| Toilet | Wound | m | 41 | 41 | 41 | 41 | 41 | 41 |
| roll | length | | | | | | | |
| | Roll weight | g | 188 | 185 | 188 | 185 | 187 | 187 |
| | (per 114 mm | | | | | | | |
| | width) | | | | | | | |
| | Wound | mm | 132 | 130 | 130 | 128 | 130 | 130 |
| | diameter | | | | | | | |
| | Outer | mm | 39 | 39 | 39 | 39 | 39 | 39 |
| | diameter of | | | | | | | |
| | paper tube | | | | | | | |
| | Winding | m/cm² | 0.66 | 0.68 | 0.68 | 0.70 | 0.68 | 0.68 |
| | density | | | | | | | |
| | Softness of | mm | 2.2 | 2.1 | 1.9 | 1.7 | 2.0 | 2.0 |
| | wound roll | | | | | | | |
| | Roll | g/cm³ | 0.132 | 0.134 | 0.137 | 0.139 | 0.136 | 0.136 |
| | density | | | | | | | |

| | | | | | | | |
|---|---|---|---|---|---|---|---|
| Evaluation | Resistance to tearing | 4 | 4 | 4 | 4 | 4 | 4 |
| | when used with warm- | | | | | | |
| | water washing toilet | | | | | | |
| | seats | | | | | | |
| | Wound length | 4 | 4 | 4 | 4 | 4 | 4 |
| | Wound diameter | 4 | 4 | 4 | 4 | 4 | 4 |
| | Ractile sensation | 3 | 4 | 4 | 3 | 4 | 4 |
| | (smoothness) | | | | | | |
| | Difference in | 4 | 4 | 3 | 3 | 4 | 4 |
| | smoothness between | | | | | | |
| | outer surface side and | | | | | | |
| | inner surface side | | | | | | |
| | Roll softness | 4 | 4 | 4 | 4 | 4 | 4 |
| | Sense of volume | 4 | 4 | 4 | 4 | 4 | 4 |
| | Softness of | 2 | 3 | 3 | 2 | 4 | 4 |
| | toilet paper | | | | | | |
| | Aesthetic appearance | 2 | 3 | 3 | 2 | 4 | 4 |
| | of embosses | | | | | | |

TABLE 3

| | Evaluation | | Comp. | Comp. | Comp. | Comp. | Comp. | Comp. | Comp. | Comp. | Comp. | Comp. |
|---|---|---|---|---|---|---|---|---|---|---|---|---|
| | items | | Ex. 1 | Ex. 2 | Ex. 3 | Ex. 4 | Ex. 5 | Ex. 6 | Ex. 7 | Ex. 8 | Ex. 9 | Ex. 10 |
| Toilet | Basis | g/m² | 20.0 | 19.8 | 19.9 | 20.1 | 14.0 | 30.0 | 19.8 | 19.9 | 20.0 | 20.1 |
| paper | weight | (1 ply) | | | | | | | | | | |
| | Paper | mm | 1.31 | 1.33 | 1.43 | 1.17 | 0.90 | 1.95 | 1.01 | 1.67 | 1.40 | 1.21 |
| | thickness | (10 plies) | | | | | | | | | | |
| | Specific | cm³/g | 6.6 | 6.7 | 7.2 | 5.8 | 6.4 | 6.5 | 5.1 | 8.4 | 7.0 | 6.0 |
| | volume | | | | | | | | | | | |
| | DMDT | N/25 mm | 4.09 | 4.13 | 4.08 | 4.08 | 2.77 | 6.20 | 4.35 | 3.71 | 3.89 | 4.37 |
| | | (product ply) | | | | | | | | | | |
| | DCDT | N/25 mm | 1.29 | 1.36 | 1.34 | 1.36 | 0.92 | 2.34 | 1.46 | 1.14 | 1.24 | 1.42 |
| | | (product ply) | | | | | | | | | | |
| | Disintegration | seconds | 45 | 46 | 46 | 44 | 20 | 145 | 46 | 42 | 33 | 58 |
| | property | (1 ply) | | | | | | | | | | |
| | Content of | % | 0.04 | 0.04 | 0.04 | 0.04 | 0.002 | 0.30 | 0.04 | 0.04 | 0.04 | 0.04 |
| | temporary | | | | | | | | | | | |
| | wet strength | | | | | | | | | | | |
| | agent | | | | | | | | | | | |
| | Depth of | mm | 0.30 | 0.32 | 0.30 | 0.31 | 0.24 | 0.40 | 0.03 | 0.53 | 0.30 | 0.31 |
| | first | | | | | | | | | | | |
| | emboss | | | | | | | | | | | |
| | (outer | | | | | | | | | | | |
| | surface | | | | | | | | | | | |
| | side): C | | | | | | | | | | | |
| | Depth of | mm | 0.15 | 0.14 | 0.15 | 0.15 | 0.08 | 0.22 | 0.02 | 0.26 | 0.14 | 0.16 |
| | second | | | | | | | | | | | |
| | emboss | | | | | | | | | | | |
| | (outer | | | | | | | | | | | |
| | surface | | | | | | | | | | | |
| | side): D | | | | | | | | | | | |
| | C − D | mm | 0.15 | 0.18 | 0.15 | 0.16 | 0.16 | 0.18 | 0.01 | 0.27 | 0.16 | 0.15 |
| | C/D | — | 2.00 | 2.29 | 2.00 | 2.07 | 3.00 | 1.82 | 1.50 | 2.04 | 2.14 | 1.94 |
| | Area ratio of | % | 66 | 66 | 67 | 66 | 66 | 66 | 66 | 66 | 10 | 100 |
| | first emboss | | | | | | | | | | | |
| | (outer | | | | | | | | | | | |
| | surface side) | | | | | | | | | | | |
| | Number of | pieces/1 | 60 | 60 | 58 | 61 | 59 | 60 | 56 | 57 | 61 | 58 |
| | embosses | cm² | | | | | | | | | | |
| | (inner | | | | | | | | | | | |
| | surface side) | | | | | | | | | | | |
| | Emboss depth | mm | 0.15 | 0.15 | 0.20 | 0.10 | 0.10 | 0.21 | 0.15 | 0.14 | 0.14 | 0.16 |
| | (inner | | | | | | | | | | | |
| | surface side) | | | | | | | | | | | |
| | TS750 (outer | dBV² | 33.2 | 33.1 | 34.8 | 31.6 | 28.8 | 37.1 | 15.4 | 54.5 | 14.0 | 54.5 |
| | surface side) | rms | | | | | | | | | | |
| | (a) | | | | | | | | | | | |
| | TS750 (inner | dBV² | 34.0 | 34.0 | 35.5 | 32.1 | 30.0 | 38.9 | 15.1 | 70.8 | 12.5 | 97.5 |
| | surface side) | rms | | | | | | | | | | |
| | (b) | | | | | | | | | | | |
| | (b)/(a) | — | 1.02 | 1.03 | 1.02 | 1.02 | 1.04 | 1.05 | 0.98 | 1.30 | 0.89 | 1.79 |
| | Ply | Glue/water | glue | glue | glue | glue | glue | glue | glue | glue | glue | glue |
| | bonding | | | | | | | | | | | |
| | Peel strength | N/76 | 0.19 | 0.18 | 0.19 | 0.19 | 0.17 | 0.18 | 0.05 | 0.36 | 0.04 | 0.42 |
| | (average) | mm | | | | | | | | | | |
| | Peel strength | N/76 | 0.41 | 0.41 | 0.42 | 0.42 | 0.40 | 0.40 | 0.17 | 0.64 | 0.33 | 0.44 |
| | (maximum) | mm | | | | | | | | | | |
| Toilet | Wound | m | 28 | 70 | 28 | 65 | 53 | 33 | 44 | 41 | 37 | 44 |
| roll | length | | | | | | | | | | | |
| | Roll weight | g | 128 | 316 | 127 | 298 | 169 | 226 | 199 | 186 | 169 | 202 |
| | (per 114 mm | | | | | | | | | | | |
| | width) | | | | | | | | | | | |
| | Wound | mm | 110 | 167 | 137 | 125 | 125 | 142 | 121 | 144 | 130 | 130 |
| | diameter | | | | | | | | | | | |
| | Outer | mm | 39 | 39 | 39 | 39 | 39 | 39 | 39 | 39 | 39 | 39 |
| | diameter of | | | | | | | | | | | |
| | paper tube | | | | | | | | | | | |
| | Winding | m/cm² | 0.67 | 0.68 | 0.41 | 1.17 | 0.96 | 0.45 | 0.85 | 0.54 | 0.61 | 0.73 |
| | density | | | | | | | | | | | |
| | Softness of | mm | 2.0 | 2.1 | 5.8 | 0.3 | 1.7 | 2.3 | 1.1 | 3.3 | 2.5 | 1.7 |
| | wound roll | | | | | | | | | | | |
| | Roll | g/cm³ | 0.135 | 0.134 | 0.082 | 0.236 | 0.134 | 0.135 | 0.169 | 0.108 | 0.123 | 0.147 |
| | density | | | | | | | | | | | |

| | | | | | | | | | | | |
|---|---|---|---|---|---|---|---|---|---|---|---|
| Evaluation | Resistance to tearing | 4 | 4 | 4 | 4 | 1 | 4 | 4 | 3 | 4 | 4 |
| | when used with warm- | | | | | | | | | | |
| | water washing toilet | | | | | | | | | | |
| | seats | | | | | | | | | | |
| | Wound length | 1 | 1 | 1 | 1 | 2 | 2 | 4 | 4 | 3 | 4 |
| | Wound diameter | 1 | 1 | 4 | 4 | 4 | 3 | 2 | 3 | 4 | 4 |
| | Ractile sensation | 4 | 4 | 4 | 4 | 4 | 4 | 1 | 1 | 1 | 1 |
| | (smoothness) | | | | | | | | | | |
| | Difference in | 4 | 4 | 4 | 4 | 4 | 4 | 3 | 3 | 2 | 1 |
| | smoothness between | | | | | | | | | | |
| | outer surface side and | | | | | | | | | | |
| | inner surface side | | | | | | | | | | |
| | Roll softness | 4 | 4 | 1 | 1 | 4 | 4 | 3 | 3 | 4 | 4 |
| | Sense of volume | 4 | 4 | 4 | 3 | 1 | 1 | 2 | 4 | 4 | 4 |
| | Softness of | 4 | 4 | 4 | 4 | 4 | 1 | 2 | 2 | 4 | 1 |
| | toilet paper | | | | | | | | | | |
| | Aesthetic appearance | 4 | 4 | 4 | 4 | 4 | 4 | 1 | 4 | 1 | 1 |
| | of embosses | | | | | | | | | | |

From the above, it was at least confirmed that, according to the present example, since a temporary wet strength agent is contained, the product is suitable for use with warm-water washing toilet seats, is resistant to tearing, excels in smoothness, aesthetic appearance of embosses, and sense of volume, and further, a toilet roll having an appropriate wound length and wound diameter and also exhibiting favorable roll softness can be obtained.

### REFERENCE SIGNS LIST

1 Toilet roll
1a Roll outer surface side
1b Roll inner surface side
1e End edge of outermost winding of toilet paper
1x Toilet paper
10, 11, 12 Emboss

1. A toilet roll comprising toilet paper,
wherein the toilet paper is formed by laminating two-ply or three-ply sheets, which have been subjected to embossing and bonded with water and/or glue, and wound into a roll shape,
the roll has a softness of wound roll of not less than 0.5 mm and not more than 4.8 mm, and a wound diameter of not less than 120 mm and not more than 150 mm,
the toilet paper contains a temporary wet strength agent, and a basis weight per ply of not less than 16 g/m² and not more than 26 g/m²,
when a sample of the toilet paper installed on a sample stage is measured using a tissue softness analyzer (TSA) by pressing a blade-equipped rotor from above with a pressing force of 100 mN and then rotating at a rotation speed of 2.0 (/sec), and a vibration of the sample stage is measured by a vibration sensor, an intensity (TS750) of the first spectral maximum peak from the low-frequency side, automatically obtained by software on the TSA, is such that a TS750 (a) on the roll outer surface side is not less than 15 dBV²rms and not more than 50 dBV²rms, a TS750 (b) on the roll inner surface side is not less than 15 dBV²rms and not more than 85 dBV²rms, and a ratio (TS750 (b)/TS750 (a)) is not less than 0.8 and not more than 1.7,
the embossing is provided on at least the ply on the roll outer surface side and includes a first emboss that is linear, and
an area ratio of the first emboss in the toilet paper is not less than 35% and not more than 93%.

2. The toilet roll according to claim 1, wherein a depth of the first emboss in the ply on the roll outer surface side is not less than 0.07 mm and not more than 0.45 mm.

3. The toilet roll according to claim 1, wherein the embossing further includes a dot-shaped second emboss, and a depth of the second emboss in the ply on the roll outer surface side is not less than 0.05 mm and not more than 0.24 mm.

4. The toilet roll according to claim 1, wherein a roll wound length is not less than 33 m and not more than 57 m.

5. The toilet roll according to claim 1, wherein a roll density is not less than 0.10 g/cm³ and not more than 0.20 g/cm³.

6. The toilet roll according to claim 1, wherein a result of a disintegration test as defined in JIS P 4501 for the toilet paper is not less than 25 seconds and not more than 100 seconds.

7. The toilet roll according to claim 1, wherein a content of the temporary wet strength agent is not less than 0.005% and not more than 0.2%.

8. The toilet roll according to claim 1, wherein the embossing is double embossing provided to the ply on the roll outer surface side and the ply on the roll inner surface side before lamination, and a number of embosses per 1 cm² in the ply on the roll inner surface side is not less than 25 and not more than 95.

9. The toilet roll according to claim 1, wherein the embossing is double embossing provided to the ply on the roll outer surface side and the ply on the roll inner surface side before lamination, and a depth of the emboss in the ply on the roll inner surface side is not less than 0.03 mm and not more than 0.35 mm.

10. The toilet roll according to claim 1, wherein the roll winding density is not less than 0.48 m/cm² and not more than 1.0 m/cm².

11. The toilet roll according to claim 1, wherein an average peel strength of the toilet paper is not less than 0.09 N/76 mm and not more than 0.32 N/76 mm.

12. The toilet roll according to claim 1, wherein a maximum value of the peel strength of the toilet paper is not less than 0.20 N/76 mm and not more than 0.60 N/76 mm.

13. The toilet roll according to claim 1, wherein a basis 5 weight of the product plies of the toilet paper is not less than 32 g/m² and not more than 52 g/m².

14. The toilet roll according to claim 3, wherein a ratio (C)/(D) of the depth (C) of the first emboss to the depth (D) of the second emboss is not less than 1.05 and not more than 8.0.

15. The toilet roll according to claim 1, wherein a roll weight is not less than 150 g and not more than 270 g.
